(19) United States
(12) Patent Application Publication
(10) Pub. No.: US 2023/0047758 A1
(43) Pub. Date: Feb. 16, 2023

(54) SURGICAL DISSECTOR INSTRUMENT

(71) Applicant: Damien Grinsell, Toorak (AU)

(72) Inventor: Damien Grinsell, Toorak (AU)

(21) Appl. No.: 17/877,146

(22) Filed: Jul. 29, 2022

(30) Foreign Application Priority Data
Jul. 30, 2021 (AU) 2021902361

(51) Int. Cl. A61B 17/32

(52) U.S. Cl. CPC A61B 17/32; A61B 2017/32004

(57) ABSTRACT

Provided is a surgical dissector instrument including a pair of arms pivotably connected intermediate their ends, with each arm having a handle at one end, and a tooth formation at the other, wherein the tooth formations are mutually opposable and asymmetrical in profile.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### RELATED APPLICATION INFORMATION

This application claims the benefit of Australian Provisional Patent Application No. 2021902361, filed on 30 Jul. 2022, the entire contents of which is incorporated herein by reference thereto.

### FIELD OF THE INVENTION

The present invention relates generally to surgical instruments. More particularly, the present invention relates to surgical dissector instruments.

It will be convenient to hereinafter describe the invention in relation to surgical dissector instruments. However, it is to be appreciated that the present invention is not limited solely to that use. For example, the surgical dissector instrument could be used in other procedures that do not involve dissection. The surgical dissector instrument could also be used in non-surgical procedures and the invention should not be construed as limited to any one or more of the specific examples provided herein.

### BACKGROUND

Any discussion of documents, devices, acts or knowledge in this specification is included to explain the context of the invention. It should not be taken as an admission that any of the material forms a part of the prior art base or the common general knowledge in the relevant art in Australia, or elsewhere, on or before the priority date of the disclosure herein.

Surgery often requires the use of specially adapted surgical instruments depending on the type of surgery. In reconstructive flap surgery (i.e. flap surgery), the flap normally needs to be excised in a single piece. This requires the blood vessels and capillaries of the flap's vascular pedicle to be bisected from the donor site to enable the flap to be removed and subsequently reattached at the recipient site. Flap surgery may generally require the use of scissors, a dissector, forceps, and an electrocauteriser, but can require the use of other surgical instruments. The techniques involved in flap surgery can include dissecting the flap tissue with the scissors, forceps, and dissector, as well as cauterising and bisecting blood vessels and capillaries. A normal procedure for cauterising and bisecting blood vessels and capillaries in flap surgery involves first cauterising the vessel or capillary in two adjacent locations and then bisecting the vessel or capillary in between the two cauterised locations. This technique of cauterising and bisecting of blood vessel and capillaries may be used in other types of surgery.

Surgery is preferably completed in the shortest time possible to avoid the risk of potential complications from arising and to keep costs to a minimum. Swapping instruments during surgery consumes time and increases the risk of potential complications. Reconstructive flap surgery, particularly, small fine dissection of flaps for microvascular transfer, plastic surgery and other types of surgery could be easier, shorter, and safer if the number of surgical instruments required could be reduced.

It would therefore be desirable to provide an instrument for performing flap surgery, in particular, small fine dissection of flaps for microvascular transfer surgery, plastic surgery, and other types of surgery which reduces the number of surgical instruments required to be used, or which at least provides a useful alternative.

### SUMMARY

Accordingly, in one aspect, the present invention may provide a surgical dissector instrument including: a pair of arms pivotably connected intermediate their ends, each arm having a handle at one end and a tooth formation at the other, wherein the tooth formations are mutually opposable.

Preferably, each of the tooth formations further includes mutually opposable contact surfaces.

Preferably, each of the mutually opposable contact surfaces further includes a distal and a proximal portion wherein the respective distal portions have smaller contact areas than the proximal portions.

Preferably, each of the mutually opposable contact surfaces are planar.

Preferably, the mutually opposable contact surfaces are smooth.

Preferably, each arm further includes a conductor extending from a proximal end of the handle, or from another suitable location, to the mutually opposable contact surfaces so that current can flow from the proximal end of the handle, or from the another suitable location, of one of the arms along that arm to its mutually opposable contact surface and electrically arc to the mutually opposable contact surface of the other arm and flow along that other arm and out of that other arm's proximal end of the handle, or from the another suitable location, to complete an electrical circuit so that the mutually opposable contact surfaces are able to cauterise a blood vessel or other body tissue.

Preferably, the mutually opposable contact surfaces are sufficiently smooth so as not to stick to a blood vessel or body tissue when used to cauterise a blood vessel or body tissue.

Preferably, the mutually opposable contact surfaces are sufficiently large to cauterise a blood vessel in a single action at a single location on the blood vessel to enable the blood vessel to be bisected at the cauterised location so as to cause effective stop of blood flow from the ends of the bisected blood vessel.

Preferably, the distal portions of each of the mutually opposable contact surfaces have profiles which further include respective apexes.

Preferably, each of the tooth formations defines a respective blunt apex and the blunt apexes co-extend with the apexes of the distal portions of the respective profiles of the mutually opposable contact surfaces.

Preferably, the profiles of the mutually opposable contact surfaces align with each other when closed together.

Preferably, the profiles of the mutually opposable contact surfaces are asymmetrical.

Preferably, the handle of each arm further includes a circular finger support aperture and the respective portions of the arms intersecting the circular finger support apertures include respective circular finger supports substantially similar or the same in shape to a portion of either or both of the circular finger support apertures.

Preferably, the arms are curvilinearly shaped at their distal ends.

Preferably, the arms include respective blades adjacent the tooth formations.

These and other essential or preferred features of the present invention will be apparent from the description that now follows.

### BRIEF DESCRIPTION OF THE DRAWINGS

In order that the invention may be more clearly understood and put into practical effect there shall now be described in detail a preferred surgical dissector instrument made in accordance with a preferred embodiment of the present invention. The ensuing description is given by way of non-limitative examples only and is with reference to the accompanying drawings, wherein:

FIG. 1 is a perspective view of a surgical dissector instrument made in accordance with a preferred embodiment of the present invention;

FIG. 2 shows a cutaway enlarged partial perspective view of the surgical dissector instrument of FIG. 1;

FIG. 3 is an underside view of the surgical dissector instrument of FIG. 1; and,

FIG. 4 is a side view of the surgical dissector instrument of FIG. 1.

### DETAILED DESCRIPTION

In the following detailed description of the invention, reference is made to the drawings in which like reference numerals refer to like elements throughout, and which are intended to show by way of illustration specific embodiments in which the invention may be practiced. It is to be understood that other embodiments may be utilized and that procedural and/or structural changes may be made without departing from the spirit and scope of the invention.

In the drawings there is shown a preferred embodiment of a surgical dissector instrument 1 made in accordance with the present invention. Surgical dissector instrument 1 is particularly suitable for reconstructive flap surgery, in particular, for small fine dissection of flaps for microvascular transfer and/or other microsurgical applications, however, it will be appreciated that surgical dissector instrument 1 is not limited to that/those uses only.

Referring to the drawings, it can be seen that surgical dissector instrument 1 has a pair of arms 2 pivotably connected intermediate their ends by way of, for example, a pin, screw or the like 4, as shown, with each arm 2 having a handle at one end in the form of a circular finger support aperture 6, a mutually opposable tooth formation 8 at the other end, and a curvilinearly shaped blade 10 adjacent the tooth formation 8. Tooth formations 8 preferably have smooth, planar, mutually opposable contact surfaces 12, with proximal 14 and distal 16 portions—the distal portions 16 having a smaller contact surface area than the proximal portions 14. Each arm 2 of surgical dissector instrument 1 includes a conductor extending from, or being, a protrusion 28 disposed adjacent to the proximal end of its respective circular finger support aperture 6, or from another suitable location (not shown), to its mutually opposable contact surface 12. Electrical power (not shown) can be applied to the protrusions 28 disposed adjacent to the proximal ends of the circular finger support apertures 6, or at other suitable locations (not shown) of each respective arm 2, to cause an electrical current to flow along one of the arms 2 to its respective mutually opposable contact surface 12, and arc across (not shown) to the mutually opposable contact surface 12 of the other arm 2, and flow along that other arm 2 to the protrusion 28 disposed adjacent to the proximal end of its circular finger support aperture 6, or to another suitable location (not shown), so that the mutually opposable contact surfaces 12 are able to cauterise a blood vessel or other body tissue (not shown).

The mutually opposable contact surfaces 12 are preferably sufficiently smooth so as not to stick to a blood vessel or capillary (not shown) when used to cauterise the same. They also preferably have a sufficiently large contact area so that they can cauterise a blood vessel or capillary in a single action at a single location including to prepare the blood vessel or capillary for bisection. When a cauterised blood vessel or capillary (not shown) is subsequently bisected at the cauterised location, blood flow from the bisected ends (not shown) will be expected to effectively stop. Because the mutually opposable contact surfaces 12 are smooth they will also be less likely to stick to or will not stick to other body tissue (not shown).

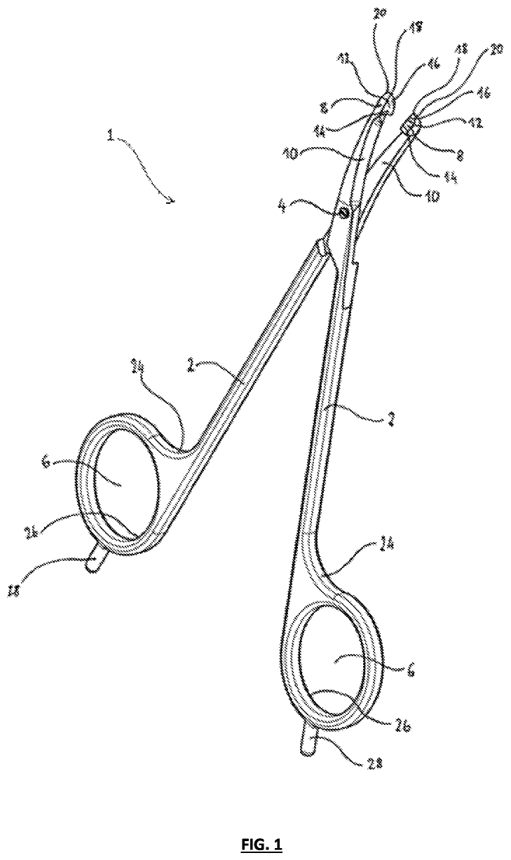

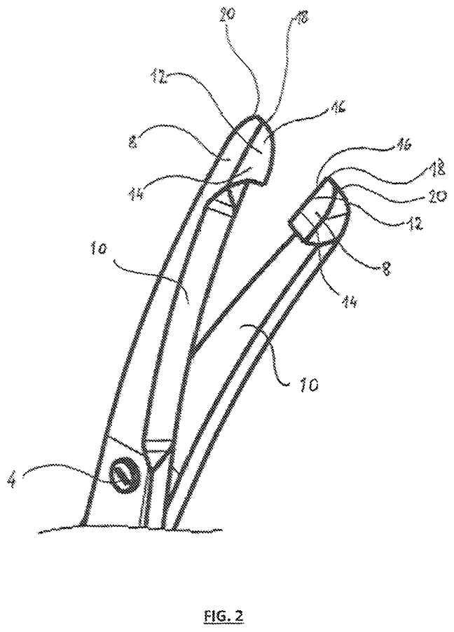

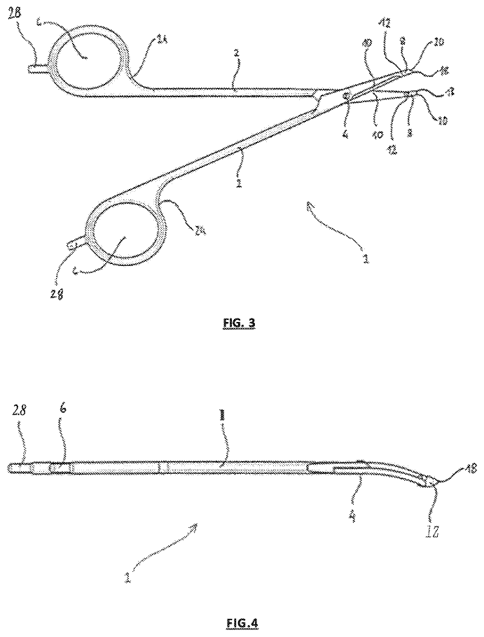

As can be best seen in FIG. 2, the profiles of the distal portions 16 of the mutually opposable contact surfaces 12 have respective apexes 18 which co-extend with respective blunt apexes 20 of the tooth formations 8. The profiles of the mutually opposable contact surfaces 12 align when closed together (as is indicated by reference number 22, in FIG. 4), giving the tooth formations 8 end of the surgical dissector instrument 1 a generally congruent shape as best shown in FIG. 4. Whilst the mutually opposable contact surfaces 12 align when closed together (at reference number 22), as can be seen in the drawings, the profiles of the mutually opposable contact surfaces 12 are preferably asymmetrical, as shown.

The portion of each arm 2 intersecting the circular finger support aperture 6 has a respective circular finger support 24 which is substantially the same in shape to a portion 26 (see, FIG. 1) of the finger support aperture 6.

In its preferred form, surgical dissector instrument 1 can be used in reconstructive flap surgery, in particular, for small fine dissection of flaps for microvascular transfer. This type of surgery involves excising a flap from a donor site for attachment to a recipient site. The respective finger support apertures 6 of each arm 2 may firstly be engaged with the fingers (not shown) into a secure hold of the surgical dissector instrument 1. If required, an initial dissection can be made in the flap donor site by, for example, using the distal portions 16 of the mutually opposable contact surfaces 12 in a clipping action to create better access to the donor site. The flap tissue is excised into the desired shape by any suitable technique including involving any one, or any suitable combination of the following: using the distal portions 16 of the mutually opposable contact surfaces 12 in a clipping action; using the planar mutually opposable contact surfaces 12 as forceps; using the apexes 18 of the mutually opposable contact surfaces or the blunt apexes 20 of the tooth formations 8 in a gouging or lacerating action (including when the mutually opposable contact surfaces are closed together—as is indicated by reference numeral 22 in FIG. 4); and/or, by using the blades 10, including in a scissor action, to make cuts.

The preferred smooth, planar surfaces of the mutually opposable contact surfaces 12 can be brought into sliding contact with the flap tissue while simultaneously cauterising it to advantageously cause the flap tissue to dissect at the site of contact. To assist with a steady and controlled motion of surgical dissector instrument 1, the fingers of a hand (not shown) can engage the circular finger supports 24 into a more secure hold. It will be appreciated that in the course of flap surgery the circular finger supports 24 require less mental adjustment while being engaged by the fingers due to their predictable shape compared to portions 26 (see, FIG. 1) of the finger support apertures 6. Surgical dissector instrument 1 can be used to dissect other tissue and in other types of surgery by using the same, similar, or another suitable technique.

A blood vessel or capillary connecting the donor site to the flap can advantageously be cauterised for dissection in a single action at a single location due to the mutually opposable contact surfaces 12 having a sufficiently large surface area and being advantageously sufficiently smooth so as not to stick to the blood vessel or capillary. A blood vessel or capillary thusly cauterised by the mutually opposable contact surfaces 12 can be bisected by the blades 10 of the arms 2 in a scissor action, where appropriate, or advantageously may be bisected by bringing the smooth, planar surfaces of the mutually opposable contact surfaces 12 into sliding contact with the blood vessel or capillary while also simultaneously cauterising it to advantageously cause the blood vessel or capillary to dissect at the site of contact, including without causing the surgical dissector instrument 1 to necessarily protrude beyond the site of contact. The distal portions 16 of the mutually opposable contact surfaces 12, being of a smaller contact area than the proximal portions 14 can be advantageously used to bisect cauterised fine blood vessels or capillaries in the same, similar, or other suitable manner. Where larger blood vessels are required to be bisected (including following cauterisation) the apexes 18 and distal portions 16 (the combination being suitable for a clipping action) can be used to first partly clip the larger blood vessel so it then can subsequently be cut in a scissor action of the blades 10, or where suitable can be used to clip and bisect the blood vessel without use of the blades 10. The blunt apexes 20 can advantageously help to protect surrounding blood vessels and capillaries or other tissue (not shown) from undesired damage in the course of excising the flap. The surgical dissector instrument 1 can be used to cauterise and bisect blood vessels and capillaries, and other body tissue in other types of surgery.

While this invention has been described in connection with specific embodiments thereof, it will be understood that it is capable of further modification. The present invention is intended to cover any variations, uses or adaptations of the invention following in general, the principles of the invention and including such departures from the present disclosure as come within known or customary practice within the art to which the invention pertains and as may be applied to the essential features hereinbefore set forth.

As the present invention may be embodied in several forms without departing from the spirit of the essential characteristics of the invention, it should be understood that the above described embodiments are not to limit the present invention unless otherwise specified, but rather should be construed broadly within the spirit and scope of the invention as defined in the attached claims. Various modifications and equivalent arrangements are intended to be included within the spirit and scope of the invention. Therefore, the specific embodiments are to be understood to be illustrative of the many ways in which the principles of the present invention may be practiced.

Where the terms “comprise”, “comprises”, “comprised” or “comprising” are used in this specification, they are to be interpreted as specifying the presence of the stated features, integers, steps or components referred to, but not to preclude the presence or addition of one or more other features, integers, steps, components to be grouped therewith.

It is claimed:

1. A surgical dissector instrument including:
a pair of arms pivotably connected intermediate their ends, each arm having a handle at one end and a tooth formation at the other end, wherein the tooth formations are mutually opposable.

2. The surgical dissector instrument as claimed in claim 1, wherein each of the tooth formations further includes mutually opposable contact surfaces.

3. The surgical dissector instrument as claimed in claim 2, wherein each of the mutually opposable contact surfaces further includes a distal and a proximal portion and the respective distal portions have smaller contact areas than the proximal portions.

4. The surgical dissector instrument as claimed in claim 2, wherein each of the mutually opposable contact surfaces are planar.

5. The surgical dissector instrument as claimed in claim 2, wherein the mutually opposable contact surfaces are smooth.

6. The surgical dissector instrument as claimed in claim 2, wherein each arm further includes a conductor extending from a proximal end of the handle, or from another suitable location, to the mutually opposable contact surfaces so that current can flow from the proximal end of the handle, or from the another suitable location, of one of the arms along that arm to its mutually opposable contact surface and electrically arc to the mutually opposable contact surface of the other arm and flow along that other arm and out of that other arm's proximal end of the handle, or from the another suitable location, to complete an electrical circuit so that the mutually opposable contact surfaces are able to cauterise a blood vessel or other body tissue.

7. The surgical dissector instrument as claimed in claim 6, wherein the mutually opposable contact surfaces are sufficiently smooth so as not to stick to a blood vessel or body tissue when used to cauterise a blood vessel or body tissue.

8. The surgical dissector instrument as claimed in claim 6, wherein the mutually opposable contact surfaces are sufficiently large to cauterise a blood vessel in a single action at a single location on the blood vessel to enable the blood vessel to be bisected at the cauterised location so as to cause effective stop of blood flow from the ends of the bisected blood vessel.

9. The surgical dissector instrument as claimed in claim 3, wherein the distal portions of each of the mutually opposable contact surfaces have profiles which further include respective apexes.

10. The surgical dissector instrument as claimed in claim 9, wherein each of the tooth formations defines a respective blunt apex and the blunt apexes co-extend with the apexes of the respective profiles of the distal portions of the mutually opposable contact surfaces.

11. The surgical dissector instrument as claimed in claim 2, wherein the profiles of the mutually opposable contact surfaces align with each other when closed together.

12. The surgical dissector instrument as claimed in claim 2, wherein the profiles of the mutually opposable contact surfaces are asymmetrical.

13. The surgical dissector instrument as claimed in claim 1, wherein the handle of each arm further includes a circular finger support aperture and the respective portions of the arms intersecting the circular finger support apertures include respective circular finger supports substantially similar or the same in shape to a portion of either or both of the circular finger support apertures.

14. The surgical dissector instrument as claimed in claim 1, wherein the arms are curvilinearly shaped at their distal ends.

15. The surgical dissector instrument as claimed in claim 1, wherein the arms include respective blades adjacent the tooth formations.
